(19) United States
(12) Patent Application Publication
(10) Pub. No.: US 2002/0115703 A1
(43) Pub. Date: Aug. 22, 2002

(54) Prevention of development of dyskinesias

(76) Inventors: Antti Haapalinna, Turku (FI); Juuso Juhila, Turku (FI); Jouni Sirvio, Kuopio (FI)

Correspondence Address: Finnegan, Henderson, Farabow,, Garrett & Dunner, L.L.P., 1300 I Street, N.W., Washington, DC, 20005-3315, US

(21) Appl. No.: 09/987,382

(22) Filed: Nov. 14, 2001

Related U.S. Application Data
(60) Provisional application No. 60/248,004, filed on Nov. 14, 2000.

(51) Int. Cl. A61K 31/4164

(52) U.S. Cl. 514/396

(57) ABSTRACT

The present invention relates to, among other things, a method for prevention or inhibition of the development of sensitization caused by the chronic use of dopaminergic agents with an alpha2-adrenoceptor antagonist or a pharmaceutically acceptable ester or salt thereof.

This application claims the benefit of priority under 35 U.S.C. § 119(e) of U.S. provisional application No. 60 248,004, filed on Nov. 14, 2000, which is specifically incorporated herein by reference in its entirety.

In one embodiment, the present invention relates to a method for preventing or inhibiting the development of sensitization caused by chronic use of dopaminergic agents. In another embodiment, the present invention relates to a method for prevention or inhibition of the development of sensitization caused by chronic use of dopaminergic agents with at least one alpha2-adrenoceptor antagonist or a pharmaceutically acceptable ester or salt thereof In this specification, “alpha” and “α” are used interchangeably.

Additional embodiments of the invention will be set forth in part in the description which follows, and in part will be obvious from the description, or may be learned by practice of the invention.

Dopamine is a neurotransmitter that influences many behavioral functions such as locomotor activity and learning and it is involved in neuropsychiatric disorders such as Parkinson's Disease and schizophrenia (Beninger 1983). Stimulants like amphetamine and cocaine enhance dopamine release in the CNS by inhibition of dopamine uptake from the synaptic cleft. When amphetamine is administered repeatedly in daily doses, the increase in motor activity is higher than after one single dose, a phenomenon that is called amphetamine sensitization. This phenomenon is associated with the development of drug dependency, but it may also be considered as a dyskinesias caused by chronic use of dopaminergic agents.

In animal models, α₂-adrenoceptor antagonists, such as idazoxan and atipamezole, are known to have therapeutic effects on the symptoms of Parkinson's Disease (PD). In animal models of PD, they also, after acute administration, potentiate the motor responses of dopaminergic agents such as apomorfine, L-3,4-dihydroxyphenyl-alanine(L-dopa) and amphetamine. In addition, in PD patients and animal models where the dyskinesias are developed after chronic administration of L-dopa, α2-adrenoceptor antagonists have decreased the dyskinesias by enhancing inhibition in so-called indirect pathways of basal ganglia which are influenced by D2 dopamine receptors (Brotchie, J. M., Parkinson's Disease Advances in Neurology, Vol. 80, Advances in Understanding the Neural Mechanisms Underlying L-Dopa-Induced Dyskinesias, Edited by Gerald M. Stern, Lippincott William & Wilkins, Philadelphia 1999). However, the most effective way to control dyskinesias in patients is to prevent their development during dopaminergic treatment. The development of dyskinesias has been proposed to involve the overactivity of so-called direct pathways of basal ganglia which are influenced by D1 dopamine receptors. According to the knowledge of the inventors, the use of at least one alpha2-adrenoceptor antagonist in the prevention of the development of dyskinesias has not been suggested or shown before.

### BRIEF DESCRIPTION OF THE DRAWINGS

FIG. 1 shows the mean 2-h overall ambulatory activity counts ±S.E.M. after six repeated administrations of D-amphetamine 2 mg/kg s.c. and the effect of atipamezole 1 mg/kg s.c. pre-treatment 20 minutes before D-amphetamine challenge, n=20-72. Groups: saline (days 1-8 saline); saline-amphetamine (days 1-7 saline and day 8 D-amphetamine); amphetamine (days 1-8 D-amphetamine); atipamezole (days 1-8 atipamezole before saline); atipamezole-amphetamine (days 1-8 atipamezole before D-amphetamine). Statistical significances: locomotor activity of the group compared to saline-saline group (***P<0.001, **P<0.01 and *P<0.05) and locomotor activity of the group compared to amphetamine-amphetamine group (⁺⁺⁺P<0.001, ⁺⁺P<0.01 and ⁺P<0.05).

FIG. 2 shows the mean 2-h overall ambulatory activity counts ±S.E.M. at day 9, n=5-29. Chronic treatment groups: saline (days 1-8 saline); atipam. (days 1-8 atipamezole 1 mg/kg); amph. (days 1-8 D-amphetamine 2 mg/kg); atipam-amph. (days 1-8 atipamezole 1 mg/kg 20 minutes before D-amphetamine 2 mg/kg). All drugs were administrated subcutaneously in volume 0.1 ml. Drug treatments at day 9: saline (saline 20 min before saline); 1 mg/kg atipam. (atipamezole 1 mg/kg 20 min before saline); 2 mg/kg amph. (saline 20 min before D-.amphetamine 2 mg/kg); 0.3 mg/kg atipam.-2 mg/kg amph. (atipamezole 0,3 mg/kg 20 min before D-amphetamine 2 mg/kg); 1 mg/kg atipam.- 2 mg/kg amph. (atipamezole 1 mg/kg 20 min before D-amphetamine 2 mg/kg); Statistical significances: locomotor activity of the group compared to saline-saline group (***P<0.001, **P<0.01and *P<0.05), locomotor activity of the group compared to amph.-2 mg/kg amph. group (^{+++P<}0.001, ^{++P<}0.01 and ^{+P<}0.05) and locomotor activity of the group compared to the chronic saline group with same drug treatment at day 9 (^{ooo}P<0.001, ^{ooo}P<0.01 and ^{o}P<0.05).

Applicants have surprisingly discovered that an α- or alpha2-adrenoceptor antagonist, atipamezole, reduces the development and expression of sensitization (motor overactivity) when given chronically in combination with a dopaminergic stimulator, D-amphetamine, in mice. Thus, alpha2-adrenoceptor antagonists, such as atipamezole, and their pharmacologically acceptable esters or salts, can be used for prevention or inhibition of development of sensitizational conditions caused by choric use of dopaminergic agents. The sensitizational conditions include, e.g., dyskinesias and psychosis developed by chronic use of dopaminergic agents such as, apomorfine, amphetamine, and L-dopa.

Nigrostriatal dopaminergic neurons from substantia nigra to the dorsal striatum are believed to be central in the modulation of extrapyramidal motor processes. This circuitry is disturbed in PD and cause symptoms typical to PD like tremor, rigidity and difficulties in the initiation of motor actions. L-dopa has been used to relieve symptoms of PD. However, many complications are observed after continuous treatment with L-dopa, of which the most common are abnormal involuntary movements called dyskinesia (Barbeau 1974). The plastic changes in dopaminergic systems controlling motor responses are thought to be responsible for the development of dyskinesia. Alpha2-adrenoceptor antagonists, such as atipamezole, are found to enhance neuronal plasticity (Puurunen K, Jolkkonen J, Sirviö J, Haapalinna A, Sivenius J. An alpha-2 adrenergic antagonist, atipamezole, facilitates behavioral recovery after focal cerebral ischemia in rats. Neuropharmacology 40: 597-606, 2001). Furthermore, the activation of D1 dopamine receptors and the blockade of alpha-2 adrenoceptors can cause the same kind of effect in the second messenger systems of basal ganglia. Thus, repeated administration of alpha2-adrenoceptor antagonist might be ineffective or even enhance the development of dyskinesias. Locomotor hyperactivity caused by chronic activation of dopaminergic transmission by amphetamine is also a dysfunction in motor activity and is also due to sensitization effects like dyskinesia seen after chronic L-dopa treatment.

The present invention provides a new solution in the pharmacotherapy of Parkinson's disease with at least one alpha2-adrenoceptor antagonist by preventing or inhibiting the development of dyskinesia caused by the chronic use of dopaminergic agents.

Alpha2-adrenoceptor antagonists of the invention include, without limitation, atipamezole, idazoxan, and efaroxan, and their analogs and pharmaceutically acceptable salts.

4-(2-ethyl-2,3-dihydro-1H-inden-2-y1)-1H-imidazole, known as atipamezole, and its pharmaceutically acceptable acid addition salts with inorganic and organic acids generally used for the purpose, are described in U.S. Pat. No. 4,689,339, which is incorporated herein by reference. The halogenated analogs of atipamezole, for example 4-(2-ethyl-5- fluoro-2,3-dihydro-1H-inden-2-y1)-1H-imidazole and 4-(2-ethyl-5,6-difluoro-2,3-dihydro-1 H-inden-2-y1)- 1H-imidazole and their pharmaceutically acceptable acid addition salts have been described in U.S. Pat. No. 5,498,623, which is incorporated herein by reference. Idazoxan, 2-(2-(1,4-benzodioxanyl))-2-imidazoline, and efaroxan, 2-(2-ethyl-2,3-dihydro-2-benzofuranyl)-4,5-dihydro- 1H-imidazole, and their pharmaceutically acceptable acid addition salts, are described in U.S. Pat. Nos. 4,818,764 and 4,411,908, respectively, both of which are incorporated herein by reference.

In one embodiment, treatment with at least one alpha2-adrenoceptor antagonist is started at the same time as the treatment with at least one dopaminergic agent. In other embodiments, the treatment or use of at least one alpha2-adrenoceptor antagonist can be started at a time before or after the treatment or use of at least one dopaminergic agent. The precise amount of the drug to be administered to a mammal according to the present invention is dependent on numerous factors known to one skilled in the art, such as, the compound to be administered, the general condition of the patient, the condition to be treated, the desired duration of use, the type of mammal, the method and route of administration etc. For example, for atipamezole given together with L-dopa, the usual daily dosage will be from 1 to 50 mg, and can be from 10 to 30 mg, divided in 1 to 4 individual doses. In another embodiment, the dose for atipamezole will be about 10 mg. In one embodiment, the alpha-2 antagonist is given simultaneously with the dopaminergic agent. Typical routes of administration include, without limitation, oral, transdermal, transmucosal, and parenteral routes.

The invention will be further clarified by the following example, which is intended to be purely exemplary of the invention, and should not be construed as limiting.

### EXAMPLE 1

The effects of atipamezole on the locomotor hyperactivity induced by repeated administration of D-amphetamine were studied in male mice.

Animals

Experiments were performed with C57BL/ 6J strain male mice from Jackson Laboratories. Mice were transferred to the laboratory at least 2 weeks prior to use. The mice were from 8 to 20 weeks of age at the beginning of an experiment. Groups of 10 mice were housed in standard polypropylene cages (38×22×15 cm) with free access to standard certified pelleted food (RM1 Maintenance Expanded SQC; Special Diet Services, Essex, UK) and water. Ambient temperature was 22±1 C.°, and a 12:12 h light dark cycle was maintained with lights on at 6 A.M. All experiments were carried out between 7 A.M. 1 and 5 P.M. The animal care was performed in accordance with International Council for Laboratory Animal Science (ICLAS) guidelines.

Drugs

D-Amphetamine sulphate (Sigma, St. Louis, Mo., U.S.A.) and atipamezole HCl (Orion Corporation, Orion Pharma, Turku, Finland) were dissolved in saline (0.9% NaCl) and administered subcutaneously (s.c.) in a 5 ml kg volume.

Motor Activity Testing

The locomotor activity of the mice was measured in transparent standard polypropylene animal cages (38×22×15 cm) with transparent covers and aspen bedding on the floors. Test cages were placed in the middle of the photobeam frame system (Photobeam Activity System PAS, Cage Rack, San Diego Instruments, San Diego, Calif.). A computer control unit registered the interruptions of photobeams from 16 individual cages. Three different types of movements were monitored: 1) ambulations (large horizontal movements), 2) fine movements (smaller horizontal movements), and 3) rearings (vertical movements). Locomotor activity was measured at 5-min intervals for 2 h immediately after D-amphetamine or saline administrations.

Sensitization schedule and atipamezole treatment

D-amphetamine was administered subcutaneously (s.c.) at a dose of 2 mg/kg. Atipamezole was administered s.c. at a dose of 1 mg/kg 20 min before locomotor activity measurement.

In the chronic treatment group, mice were administered the dose during eight days to elicit provoked locomotor hyperactivity to D-amphetamine and the effect of the atipamezole to the locomotor activity. Mice groups in the chronic treatment schedule were saline, saline-amphetamine, amphetamine, atipamezole and atipamezole-amphetamine. A day before experiment, mice were habituated to the test environment. Test groups with different drug treatments were administrated during four consecutive days. At days five and six, there were no drug administrations nor motor activity testing. At days seven and eight, the produced locomotor hyperactivity and effect of a single exposure of D-amphetamine (saline-amphetamine-group) were analysed. (Table 1).

TABLE 1
Chronic treatment

| | | saline- | | | atipamezole- |
|---|---|---|---|---|---|
| Time | saline | amphetamine | amphetamine | atipamezole | amphetamine |
| Habituation | saline | saline | saline | saline | saline |
| Day 1 | saline | saline | amph. | atipam. | atipam. and amph. |
| Day 2 | saline | saline | amph. | atipam. | atipam. and amph. |
| Day 3 | saline | saline | amph. | atipam. | atipam. and amph. |
| Day 4 | saline | saline | amph. | atipam. | atipam. and amph. |
| Day 5 | no injection | no injection | no injection | no injection | no injection |
| Day 6 | no injection | no injection | no injection | no injection | no injection |
| Day 7 | saline | saline | amph. | atipam. | atipam. and amph. |
| Day 8 | saline | amph. | amph. | atipam. | atipam. and amph. |

At day nine, the effect of different atipamezole and amphetamine administrations to the locomotor activity on the chronic treatment groups were analysed. Treatments included saline, 1 mg/kg atipamezole, 2 mg/kg D-amphetamine, 0,3 mg/kg atipamezole- 2 mg/kg D-amphetamine and 1 mg/kg atipamezole- 2 mg/kg D-amphetamine. Chronic treatment groups were saline-, atipamezole-, amphetamine- and atipamezole-amphetamine groups. Chronic groups were treated following the schedule in Table 2.

TABLE 2
Drug treatments at day 9

| | Chronic group | | | |
|---|---|---|---|---|
| | | | amphet- | atipamezole- |
| Drug treatment | saline | atipamezole | amine | amphetamine |
| saline | Yes | No | No | No |
| 1 mg/kg atipamezole | Yes | Yes | No | No |
| 2 mg/kg amphetamine | Yes | Yes | Yes | Yes |
| 0,3 mg/kg atipamezole- | | | | |
| 2 mg/kg amphetamine | Yes | No | Yes | No |
| 1 mg/kg atipamezole- | | | | |
| 2 mg/kg amphetamine | Yes | No | Yes | Yes |

All data are presented as mean ±SEM. Statistical analysis were performed using SPSS 9.0 for Windows (SPSS, Chicago, Ill.). Separate repeated measure analyses of variance (ANOVA) were performed on each variable for each experiment grouped on time and treatment group. Results were analysed separately, because data were collected in separate experiments with different study design. When significance (P<0.05) between treatment groups was found, comparisons at each time point (date or min) were analysed by using LSD post-hoc test.

RESULTS

Locomotor activity

Effect of repeated administration of D-amphetamine and atipamezole in chronic treatment groups

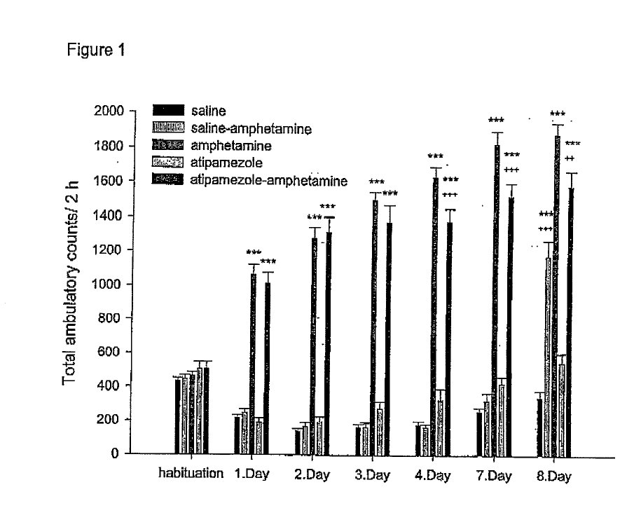

FIG. 1 illustrates the development of behavioral sensitization after six repeated administrations of D-amphetamine (2 mg/kg) and the effect of atipamezole (1 mg/kg) pre-treatment 20 minutes before D-amphetamine challenge in mice. There was a significant difference between the chronic treatment groups [F(1,184)=1618.9, P<0.001]. The activity counts were dependent on the administration Day [F(6,1104)=107.7, P<0.001] and there was a significant interaction between Day X Group [F(24,1104)=53.2, P<0.001]. Mice treated with D-amphetamine of six consecutive days (group amphetamine-amphetamine) showed a progressive enhance in ambulatory activity compared to saline group. At Day eight, mice from group saline-amphetamine were also administered with D-amphetamine, but there was still a significant difference between group amphetamine to groups saline and saline-amphetamine(P<0.001).

Atipamezole pre-treatment before saline challenge tended to enhance locomotor activity after daily atipamezole for six days, but there was not a statistically significant difference between the groups atipamezole-saline and saline at Day eight (P=0.43).

Atipamezole pre-treatment before D-amphetamine administration over the course of exposure decreased significantly D-amphetamine-induced locomotion in mice. There was a statistically significant difference between groups amphetamine-amphetamine and atipamezole-amphetamine at Day 2 (P<0.05) and Days 3-8 (P<0.001). This indicates that atipamezole pre-treatment prevented D-amphetamine-induced locomotor hyperactivity over the course of the exposure.

Effect of the different drug treatments at Day 9

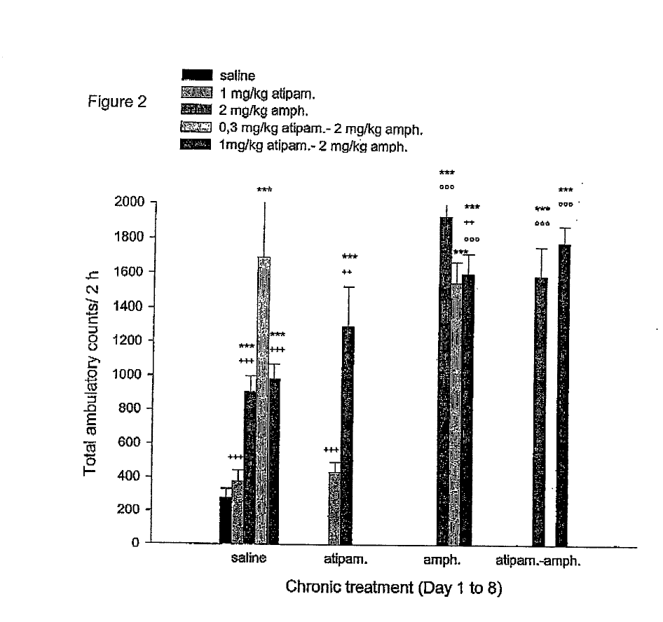

FIG. 2 illustrates the effect of the different drug treatments in chronic treatment groups at Day 9. Atipamezole pre-treatment alone did not alter total ambulatory activity counts in the saline group (P=0.81). D-amphetamine significantly enhanced locomotor activity in the saline group (P<0.001), but the activity was not the same level compared to the amphetamine group (P<0.001). Atipamezole (0.3 mg/kg) pre-treatment 20 minutes before D-amphetamine challenge amplified locomotor activity (P=0.016), but with a dosage of 1 mg/kg had no effect compared D-amphetamine alone (P=0.364). (FIG. 2, Group saline).

Atipamezole (1 mg/kg) pre-treatment over the course of exposure had a tendency to elevate locomotor activity before saline and D-amphetamine challenge, but the elevation was not statistically significant (FIG. 2, Group atipam.).

Mice in Group amphetamine developed strong ambulatory sensitization during repeated administration of D-amphetamine (2 mg/kg) (P<0.001, FIG. 2). Atipamezole doses (0.3 and 1 mg/kg) both clearly attenuated the D-amphetamine-induced locomotor hyperactivity (0.3 mg/kg P=0.005; 1 mg/kg P<0.001).

Atipamezole pre-treatment (1 mg/kg) 20 min before D-amphetamine administration over the course of exposure (group atipamezole-amphetamine) decreased significantly D-amphetamine induced locomotion. There was a statistically significant difference between groups amphetamine-amphetamine and atipamezole-amphetamine also at Day 9 (P<0.001). Part of mice from group atipamezole-amphetamine were administrated only with D-amphetamine (2 mg/kg) at Day 9 and there was still a statistically significant difference compared to amphetamine-amphetamine-group (P=0.026). This latter result is important as it shows that atipamezole causes a true reduction in the development of sensitisation, not just the |suppression of this expression.

Those skilled in the art will recognize that while specific embodiments have been illustrated and described, various modifications and changes may be made without departing from the spirit and scope of the invention.

Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

We claim

1. A method for preventing or inhibiting the development of sensitization caused by chronic use of at least one dopaminergic agent, comprising administering to a mammal in need of the preventing or inhibiting at least one alpha2-adrenoceptor antagonist in an amount effective for the preventing or inhibiting.

2. The method according to claim 1, wherein the sensitization comprises dyskinesia seen in Parkinson's Disease after chronic treatment with L-dopa.

3. The method according to claim 1, wherein the at least one alpha2-adrenoceptor antagonist comprises one or more of atipamezole and pharmaceutically acceptable salts thereof.

4. The method according to claim 1, wherein the at least one alpha2-adrenoceptor antagonist comprises one or more of idazoxan and pharmaceutically acceptable salts thereof.

5. The method according to claim 1, wherein the at least one alpha2-adrenoceptor | antagonist comprises one or more of efaroxan and pharmaceutically acceptable salts thereof.

6. The method according to claim 1, wherein the at least one alpha2-adrenoceptor antagonist comprises one or more of 4-(2-ethyl-5-fluoro-2,3-dihydro-1H-inden-2-y1)-1H-imidazole and pharmaceutically acceptable salts thereof.

7. The method according to claim 1, wherein the at least one alpha2-adrenoceptor antagonist comprises at least one analog chosen from analogs of atipamezole, analogs of idazoxan, and analogs of efaroxan.

8. The method according to claim 1, wherein the at least one alpha2-adrenoceptor antagonist comprises at least one ester chosen from esters of atipamezole, esters of idazoxan, and esters of efaroxan.

9. A method for preventing or inhibiting the development of sensitization associated with the development of drug dependency, comprising administering to a mammal in need of the preventing or inhibiting at least one alpha2-adrenoceptor antagonist in an amount effective for the preventing or inhibiting.

10. A method for preventing or inhibiting the development of sensitisation caused by at least one dopaminergic agent administered at least twice, comprising administering to a mammal in need of the preventing or inhibiting at least one alpha2-adrenoceptor antagonist in an amount effective for the preventing or inhibiting.
